(19) United States
(12) Patent Application Publication
(10) Pub. No.: US 2026/0069855 A1
(43) Pub. Date: Mar. 12, 2026

(54) BLOOD PUMP DRIVE SHAFT AND MOTOR COOLING SYSTEM

(71) Applicant: CARDIOVASCULAR SYSTEMS, INC., St. Paul, MN (US)

(72) Inventors: Tristan Van de Moortele, Minneapolis, MN (US); Grant A. Adams, New Brighton, MN (US); Matthew W. Tilstra, Rogers, MN (US); Joseph P. Higgins, Wayzata, MN (US)

(21) Appl. No.: 19/106,053

(22) PCT Filed: Jul. 13, 2023
(86) PCT No.: PCT/US2023/070112

§ 371 Date: Feb. 24, 2025

Related U.S. Application Data
(60) Provisional application No. 63/374,110, filed on Aug. 31, 2022.

(51) Int. Cl. A61M 60/802; A61M 60/174; A61M 60/216; A61M 60/414; A61M 60/515; A61M 60/538

(52) U.S. Cl. CPC A61M 60/802; A61M 60/174; A61M 60/216; A61M 60/414; A61M 60/515; A61M 60/538

(57) ABSTRACT

A cooling system for a rotational medical device such as a blood pump comprising an electric motor, a rotational drive shaft and an impeller. The cooling system comprises at least one active fluid pump for moving fluid distally within an outer fluid flow channel to a point proximal of the drive shaft's connection with the impeller where the fluid is deflected and urged into a fluidly connected inner fluid flow channel. A portion of the fluid in the outer fluid flow channel is pumped toward the impeller and the remaining fluid flows proximally through the inner fluid flow channel and along the outer surface of the drive shaft, thereby removing frictional heat from the rotating drive shaft. Next, the fluid is urged to bathe the outer surface of the electric motor to remove heat from the motor. Finally, the heated fluid is removed from the system.

### CROSS-REFERENCE TO RELATED APPLICATIONS

This application claims priority to Provisional Application Serial No. 63/374110, filed Aug. 31, 2022 and entitled BLOOD PUMP MOTOR COOLING SYSTEM, the entire contents of which are incorporated herein by reference.

### STATEMENT REGARDING FEDERALLY SPONSORED RESEARCH OR DEVELOPMENT

Not Applicable

### BACKGROUND OF THE INVENTION

### Field of the Invention

The invention relates to an intravascular pump with housing region that may be expandable and comprises an integrated lead trough defined by the housing region and extending along at least a portion of the length of the housing region.

### Description of the Related Art

With reference to FIG. 1, the human heart comprises four chambers and four heart valves that assist in the forward (antegrade) flow of blood through the heart. The chambers include the left atrium, left ventricle, right atrium and left ventricle. The four heart valves include the mitral valve, the tricuspid valve, the aortic valve and the pulmonary valve.

The mitral valve is located between the left atrium and left ventricle and helps control the flow of blood from the left atrium to the left ventricle by acting as a one-way valve to prevent backflow into the left atrium. Similarly, the tricuspid valve is located between the right atrium and the right ventricle, while the aortic valve and the pulmonary valve are semilunar valves located in arteries flowing blood away from the heart. The valves are all one-way valves, with leaflets that open to allow forward (antegrade) blood flow. The normally functioning valve leaflets close under the pressure exerted by reverse blood to prevent backflow (retrograde) of the blood.

Thus, as illustrated, the general blood flow comprises deoxygenated blood returning from the body where it is received by the right atrium via the superior and inferior vena cava and is, in turn, pumped into the right ventricle, a process controlled by the tricuspid valve. The right ventricle functions to pump the deoxygenated blood to the lungs via the pulmonary arteries, where the blood is reoxygenated and returned to the left atrium via the pulmonary veins.

Heart disease is a health problem with a high mortality rate. The use of temporary mechanical blood pump devices are used on an increasingly frequent basis to provide short-term acute support during surgery or as temporary bridging support to help a patient survive a crisis. These temporary blood pumps have developed and evolved over the years to supplement the pumping action of the heart on a short-term basis and supplement blood flow as either left or right ventricular assist devices, with the left ventricular assist device (“LVAD”) currently the most commonly used device.

Known temporary LVAD devices generally are delivered percutaneously, e.g., through the femoral artery, to locate or position the LVAD inlet in the patient's left ventricle and the outlet in the patient's ascending aorta with the body of the device disposed across the aortic valve. As the skilled artisan will understand, an incision may be made below the patient's groin to enable access to the patient's femoral artery. The physician may then translate guide wire, followed by a catheter or delivery sheath, through the femoral artery and descending aorta until reaching the ascending aorta. The LVAD with attached rotational drive shaft may then be translated through the delivery catheter or sheath lumen, leaving a proximal end of the drive shaft exposed outside of the patient and coupled with a prime mover such as an electric motor or the equivalent for rotating and controlling the rotational speed of the drive shaft and associated LVAD impeller.

Temporary axial flow blood pumps consist generally of two types: (1) those that are powered by a motor integrated into the device that is connected with the pump's impeller (see U.S. Pat. Nos. 5,147,388 and 5,275,580) ; and (2) those that are powered by an external motor that provides rotational torque to a drive shaft which is, in turn, connected to the pump's impeller (see U.S. Pat. No. 4,625,712 to Wampler and U.S. Pat. No. 5,112,349 to Summers, each hereby incorporated by reference in their entirety).

Known temporary ventricle assist devices (“VAD”), including LVAD and RVAD (right ventricular assist) devices of the present invention comprises external motor, generally comprise the following elements mounted within a housing, listed in order from the inflow end to the outflow end: an inflow aperture(s); a stationary inducer, also known as a flow straightener; a rotational impeller; and a stationary diffuser and/or outflow structure; and an outflow aperture(s).

FIG. 2 illustrates an exemplary blood pump procedure, which may comprise an LVAD or an RVAD procedure. As shown, the handle or controller comprises an electric motor therein which is rotatably connected with a rotational drive shaft extending distally away from the electric motor and which is disposed within a sheath or catheter. As seen in FIG. 3, a blood pump cannula housing may be provided. A rotatable impeller is disposed within the blood pump cannula housing, wherein the impeller is rotatably connected at its proximal end with the rotatable drive shaft. The blood pump cannula housing and drive shaft structure is translated over a pre-positioned guide wire (not shown but as well known in the art) within a patient's vasculature.

FIG. 3 illustrates an exemplary blood pump cannula housing structure positioned with inflow apertures located within a patient's left atrium and outflow apertures through the blood pump cannula housing located on the downstream side of the patient's aortic valve. The impeller (not shown but as well known in the art) is rotated by actuation of an electric motor which rotates the drive shaft and the impeller, causing blood to flow into the inflow apertures and out through the outflow apertures as shown.

Electrical power is supplied to the electric motor, e.g., a brushless DC motor, in order to cause rotation of the drive shaft and blood pump impeller. The byproduct of rotation of the electric motor is heat due to current flow through the motor windings and losses associated with the operation of the electric motor.

The electric motor, located within the controller or handle, in the exemplary VAD device comprises a power density that is great enough that cooling the motor becomes a safety risk to patient's and/or the operator. In order to eliminate this safety risk, the exterior surface of any patient-contacting device or structure is preferably maintained under 41 degrees C when the patient contact is greater than 10 minutes. In order to achieve a temperature that is below 41 degrees C, active cooling may be employed.

The present disclosure addresses, inter alia, these issues.

### BRIEF DESCRIPTION OF THE DRAWINGS

The inventions will be further described with reference to the accompanying drawings which illustrate exemplary embodiments and are not limiting.

FIG. 1 illustrates a cutaway view of the human heart.

FIG. 2 illustrates an exemplary intravascular procedure.

FIG. 3 illustrates a cutaway view of the human heart with a blood pump in position.

FIG. 4 illustrates a block diagram of one embodiment of a disclosed device.

FIG. 5A illustrates a side cutaway view of one embodiment of a disclosed device.

FIG. 5B illustrates a side cutaway view of one embodiment of a disclosed device.

FIG. 6 illustrates a longitudinal cross-sectional view of the device of FIG. 5A at cross-sectional line “A”.

FIG. 7 illustrates a block diagram of one embodiment of the disclosure.

FIG. 8 illustrates a cross-sectional, cutaway view of one embodiment of a disclosed device.

FIG. 9 illustrates a block diagram of one embodiment of the disclosure.

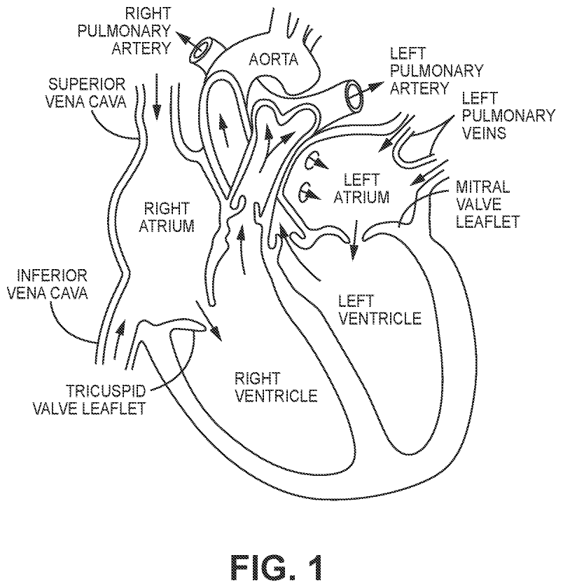

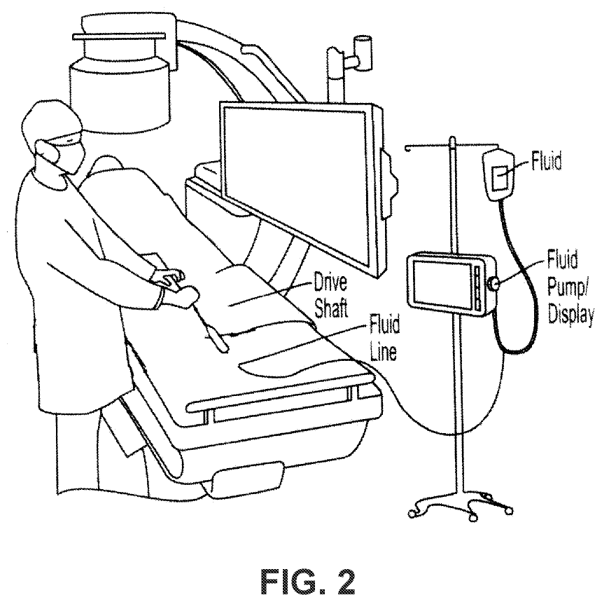

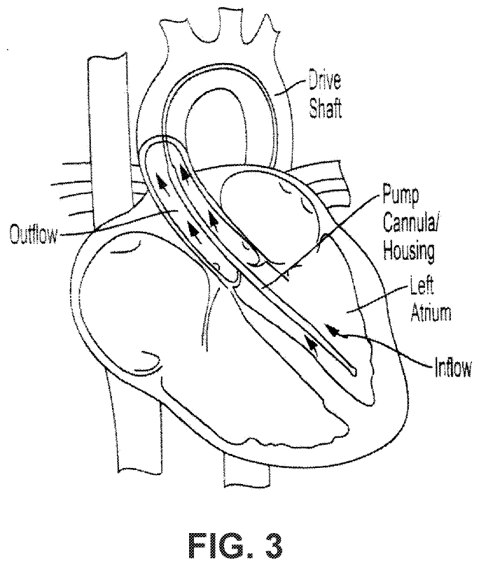

### DETAILED DESCRIPTION

While the invention is amenable to various modifications and alternative forms, specifics thereof are shown by way of example in the drawings and described in detail herein. It should be understood, however, that the intention is not to limit the invention to the particular embodiments described. On the contrary, the intention is to cover all modifications, equivalents, and alternatives falling within the spirit and scope of the invention.

Embodiments of the present invention comprises cooling the electric motor using an external water jacket, or annular structure, wherein fluid is pumped through the water jacket or annulus to carry excess heat generated by the electric motor away from the patient-contacting surfaces of the device and, thereby away from the patient.

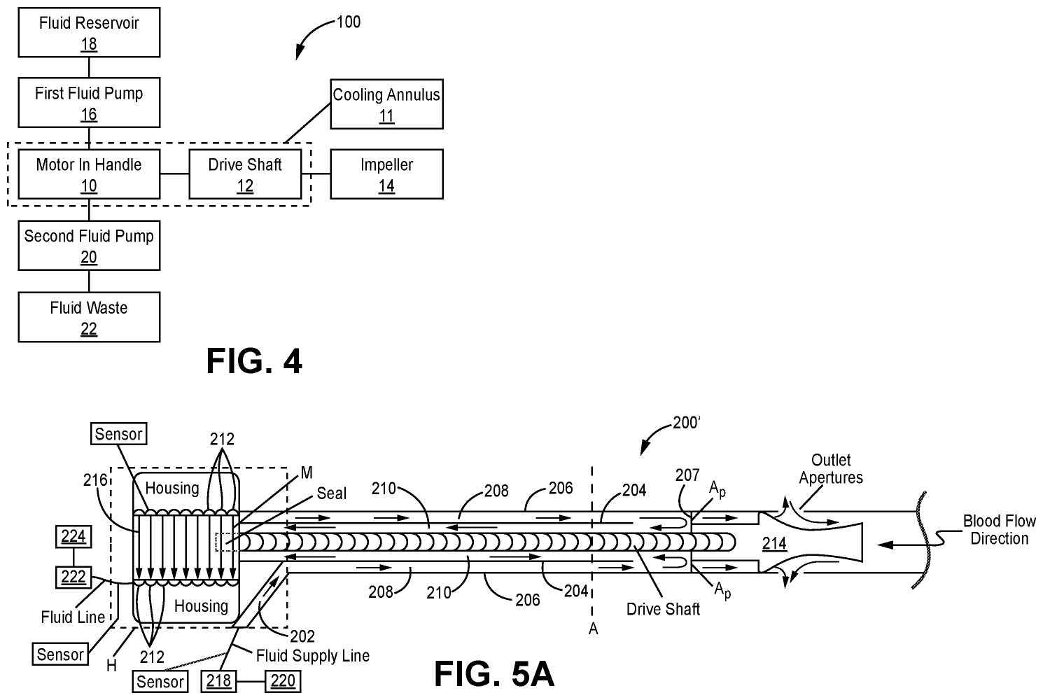

FIG. 4 illustrates a block diagram of an exemplary blood pump system 100 with a cooling feature configured to cool the blood pump motor. A handle (with integrated motor) 10 is provided with operatively attached drive shaft 12 interposed between the handle/motor 10 and a rotational blood pump impeller 14. A first fluid pump 16 is shown in fluid communication with a fluid reservoir 18. A fluid jacket or cooling annulus 11 is shown surrounding at least a portion of the motor 10 and the drive shaft 12. The first fluid pump 16 is configured to actively pump fluid from the fluid reservoir 18 into the cooling annulus. A second fluid pump 20 in fluid communication with the handle/motor 10, drive shaft 12, and/or cooling annulus 11 is also provided to actively remove the fluid after circulation through the cooling annulus 11 and with subsequent disposal of the removed fluid as fluid waste 22.

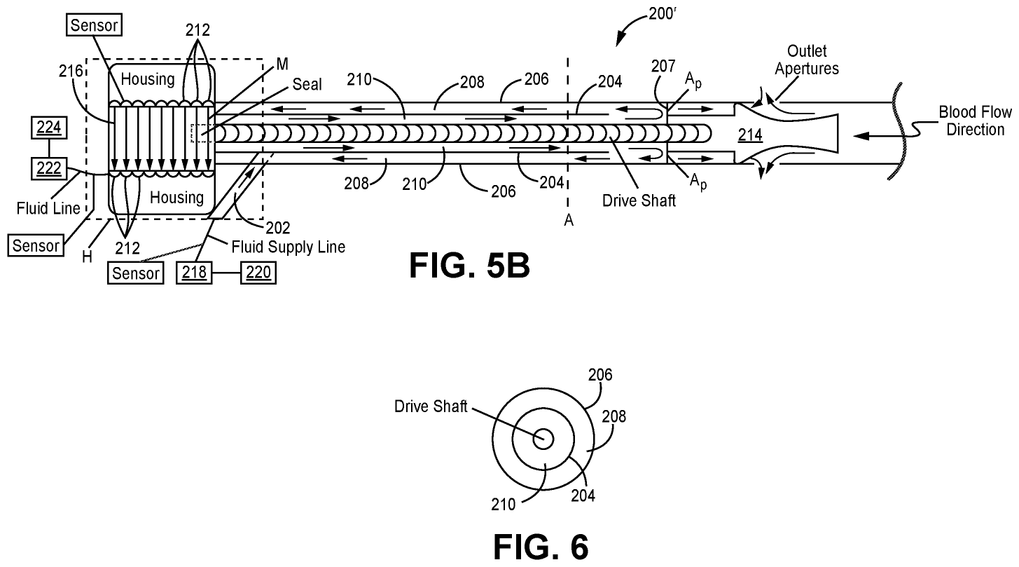

FIG. 5A provides a side cross-sectional view of one embodiment of a blood pump with a cooling mechanism 200. A handle H is provided with an electric motor M located within handle H that is defined at least in part by housing. A drive shaft is rotationally connected with motor M at a proximal end. A distal region of the drive shaft is rotationally connected with impeller 214, wherein the motor in combination with the drive shaft is configured to rotate impeller 214. The rotating impeller 214 urges fluid in a generally distal direction and out of the device through outlet apertures and into the patient's blood vessel.

A first fluid pump 218 is provided in fluid communication with a fluid reservoir 220 and, via a fluid supply line, with an inflow channel 202 within the handle H or housing. Inflow channel 202 is in fluid communication with an outer fluid flow channel 208. The outer fluid flow channel 208 is defined by an outer sheath 206 and an inner sheath 204, and is configured to provide a conduit for moving fluid in a distal direction.

Inner sheath 204 is disposed within a lumen created by outer sheath 206, wherein the outer sheath 206 and the inner sheath 204 are radially spaced from each other. The space between an inner surface of outer sheath 206 and an outer surface of inner sheath 204 comprises the outer fluid flow channel 208. The inner sheath 204 also defines an inner fluid flow channel 210 between the inner sheath 204 and the drive shaft and is configured to receive fluid from the outer fluid flow channel 208 and serve as a conduit to move the fluid in a proximal direction back toward the handle H and motor. The drive shaft rotates within a lumen defined by the inner sheath 204.

FIG. 6 illustrates a cross-sectional view along the vertical dotted line “A”, illustrating the outer sheath 206, the inner sheath 204 and the outer fluid flow channel 208 defined therebetween. FIG. 6 further illustrates the drive shaft disposed within a lumen defined by the inner sheath 204 and the inner fluid flow channel 210 defined between the drive shaft and the inner sheath 204.

The outer fluid flow channel 208 extends in the illustrated embodiment of FIG. 5A distally to a fluid deflection surface 207 that may, in some embodiments, be formed as a distal and substantially vertical wall that at least partially closes off the outer fluid flow channel 208 on the distal end. Fluid deflection surface 207 is defined at a point that is proximal to the connection of the drive shaft with the impeller 214.

Further, the inner sheath 204 extends distally, stopping at a point that is proximal to the fluid deflection surface 207 in order to provide transitional space for the deflected fluid to begin flowing back in the proximal direction through the inner fluid flow channel 210.

Arrows indicate the actively pumped fluid from first fluid pump 218 through inflow channel 202 and distally through the outer fluid flow channel 208 until the fluid is deflected by the fluid deflection surface 207 such that the fluid begins a proximal flow through the inner fluid flow channel 210. Thus, after encountering the fluid deflection surface 207, the fluid is deflected and urged into the inner fluid flow channel 210 and moves proximally toward the handle H and motor.

The fluid flowing through the inner fluid flow channel 210 absorbs heat from the rotating drive shaft and is ultimately removed from the system after absorbing heat from the electric motor M as further described below.

As shown in FIG. 5A, a substantially vertically oriented fluid deflection surface 207 may be provided, wherein the fluid deflection surface 207 is substantially orthogonal to the outer and inner fluid flow channels 208, 210.

An alternate embodiment may comprise at least part of the fluid deflection surface 207 comprising a concave shaping, or curved surface, on the proximal side. The resulting concave or curved proximal surface of the fluid deflection surface 207 may more efficiently deflect the incoming fluid from the outer fluid flow channel 208 into the inner fluid flow channel 210.

In a preferred embodiment, at least one aperture Ap may be defined through the fluid detection surface 207 to allow a portion or fraction of the incoming fluid from the outer fluid flow channel 208 to flow through the at least one aperture Ap. The at least one aperture Ap may be in fluid communication with the distally positioned impeller which may be, in turn, in fluid communication with the flowing blood within a patient's blood vessel. The impeller is configured to move fluid in a generally distal direction, out of one or more outlet apertures and into the patient's blood vessel.

The magnitude of the pressure of the fluid flow through the at least one aperture Ap is configured to prevent retrograde fluid flow, e.g., blood flow in a proximal direction, through the at least one aperture Ap. Thus, in one embodiment, the pressure of the fluid flowing through the at least one aperture Ap may be configured to have a positive pressure relative to the patient's blood pressure. Stated differently, the pressure of the fluid flowing through the at least one aperture Ap is preferably greater than the blood pressure of the patient. The resulting positive pressure configuration prevents blood from flowing from the patient's bloodstream into the cooling mechanism 200 and into the motor. Instead, as the fluid flows distally away from the at least one aperture Ap with a pressure greater than the patient's blood pressure, it will be urged forward by the impeller 214 and ultimately will flow out of the system through one or more outflow apertures and into the patient's bloodstream.

In other embodiments, the at least one aperture Ap may comprise a one-way valve that allows fluid to flow therethrough in a distal direction, but closes to prevent flow through the valve in a retrograde or proximal direction.

Continuing with reference to FIG. 5A, as the proximally flowing fluid reaches the handle H and/or housing, the fluid is flowed around the outer surface of the motor M to provide cooling for the motor M. As shown, housing H comprises a plurality of fluid guidance apertures 212 that are in fluid communication with the inner fluid flow channel 210 and located adjacent the motor M surface. As the incoming fluid from the inner fluid flow channel 210 is received within one or more of the fluid guidance apertures 212, the fluid flows 216 across the surface of motor M within the fluid guidance apertures 212, where it absorbs heat from motor M and is eventually removed from the system as will be discussed further infra. A variety of fluid flow patterns across the motor M surface may be induced or generated, including vertical and/or angled or helical. In addition, the width of the fluid guide apertures 212 may be substantially equivalent, or may vary. Further, the spacing of adjacent fluid guide apertures 212 from each other may be substantially equivalent or may be varied. Fluid flow patterns such as a helical flow pattern, as opposed to vertical, patterns provide more exposure time for the flowing fluid with the surface of the motor M and, therefore, capable of absorbing and removing more motor heat than, e.g., a vertical pattern of fluid flow. In addition, in certain embodiments, the fluid guidance apertures 212 may not be present so that the fluid may flow across the motor M surface without a specifically guided pattern of fluid flow.

FIG. 5A also shows the presence of optional sensors that may be located (1) at or adjacent the motor M surface and/or one or more of the fluid guidance apertures 212 and/or along the flow of the flowing fluid as it passes over or across the surface of the electric motor M; (2) along the fluid supply line that is fluidly connected with the fluid reservoir 218; and/or (3) along the waste fluid line that is in fluid communication with a second fluid pump 222.

The sensor(s) may comprise pressure, temperature, optical clarity, osmolarity, resistance and/or conductivity sensors. For example, a temperature sensor may be employed which, if temperature is sensed above a threshold limit, the purge or flow rate of the cooling fluid may be increased by increasing the discharge pressure or flow rate of the first fluid pump 218 and/or increasing the suction pressure or flow rate of the second fluid pump 222. Alternatively, if the temperature is sensed below a safe threshold limit, the purge or flow rate of the cooling fluid may be decreased by decreasing the discharge pressure or flow rate of the first fluid pump 218 and/or increasing the suction pressure or flow rate of the second fluid pump 222.

Similarly, a pressure sensor(s) may be placed in the cooling fluid flow path wherein the pressure(s) and/or flow rate(s) of the first and/or second fluid pumps are adjusted as discussed above based on the sensed pressure within the fluid flow pathway.

FIG. 5A also shows in dashed lines the location of a seal that is provided on the distal side of the motor M. The distal motor fluid seal may be a high-speed seal and is configured to prevent fluid from entering the motor M at the distal side of the motor M. If fluid is not prevented from entering the motor M, motor current may increase and provide, among other things, unpredictable current waveforms.

As briefly discussed above, and as shown in both FIGS. 4 and 5A, a second fluid pump 222 may be provided in fluid communication with the heated fluid from the motor M and configured to provide a suction pressure that pulls the heated fluid out of the housing and into a waste receptacle 224. Thus, two fluid pumps 218, 222 may be provided in a preferred embodiment, wherein the two pumps 218, 222 work in concert to actively move the fluid through the cooling mechanism 200, across the motor M surface and out of the cooling mechanism 200. In some embodiments one fluid pump, either first fluid pump 218 or second fluid pump 222 may be required. Among other things, the second fluid pump 222, as it pulls the cooling fluid out of the system may reduce fluid pressure on the distal motor fluid seal discussed above. If two pumps are provided, the first fluid pump 218 may push or infuse fluid into the cooling mechanism 200 and the second fluid pump 222 may pull fluid out of the cooling mechanism 200.

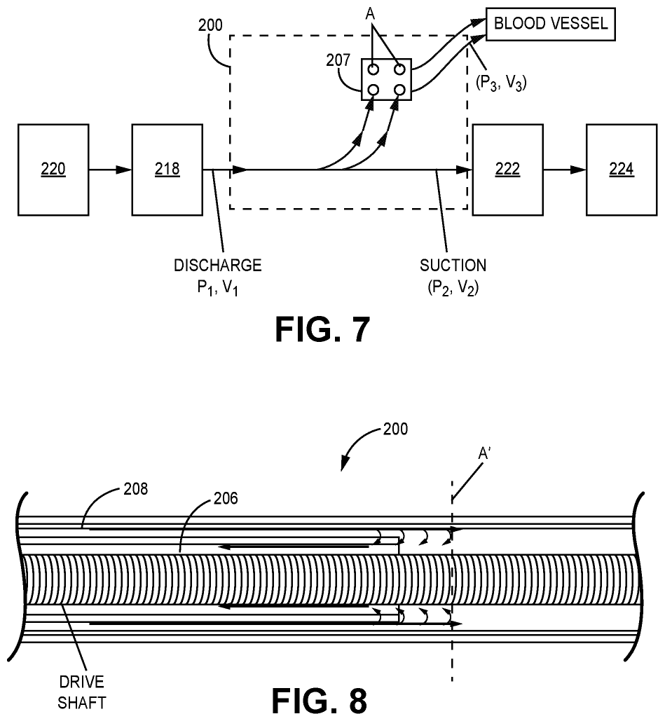

As shown in the block diagram of FIG. 7, the first fluid pump 218 may draw fluid from a reservoir 220 to provide a discharge or inflow pressure P1 and related inflow volume V1 and flow rate into the cooling mechanism 200. The second fluid pump 222 may provide a suction pressure P2 and a suction or outflow volume V1 and flow rate, wherein the second fluid pump is pulling fluid out of the cooling mechanism 200 and discharging the heated waste fluid into a waste reservoir 224. In addition, as discussed above, a portion or fraction of the fluid flowing through the outer fluid flow channel of the cooling mechanism 200 is directed out of the cooling mechanism, to the impeller and into the blood vessel. The pressure and volume of the fluid flowing through the at least one aperture Ap of the fluid deflection surface 207 may be P3 and V3 (and flow rate), respectively.

In some embodiments, the discharge or inflow pressure P1, and related inflow volume V1 or flow rate of the first fluid pump 218 may be greater than the suction or outflow pressure P2 and related suction or outflow volume V2 or flow rate generated by the second fluid pump. In this embodiment, the fluid pressure of the fluid flowing distally out of the one or more apertures A, and into the blood vessel, may be P3, where P3=P1−P2. Further, the volume of the fluid flowing distally out of the one or more apertures A, and into the blood vessel, may be V3, where V3=V1−V2. Similarly, the flow rate of the fluid flowing distally out of the one or more apertures may be (first pump 218 flow rate)−(second pump 222 flow rate)=flow rate through the one or more apertures A.

In some embodiments as discussed above and as shown in FIG. 7, a fluid deflection surface 207 may be provided as describe above to turn the flowing fluid back proximally, while also allowing some fractional volume of the fluid to move through one or more apertures Ap defined by and through the fluid deflection surface 207 as discussed above.

However, as illustrated in FIG. 8, and with continued reference to FIG. 5A, in other embodiments, the fluid deflection surface 207 may be eliminated while retaining the transition of fluid flow from the outer fluid flow channel 208 (distal flow) to the inner fluid flow channel 210 (proximal flow). The vertical dashed line A′ of FIG. 5A illustrates the location of the transition of the fluid flow from distal to proximal. The fluid deflection surface 207 described above is, however, not present in the embodiment of FIG. 8 to cause the transition of fluid flow direction. Instead, in the embodiment of FIG. 8, the first and second fluid pumps 218, 222, as described above, may be configured to generate pressures P1, P2, and related fluid volumes V1, V2 that induce the fluid flow through the cooling mechanism 200 in a first distal direction through the inner fluid flow channel 210. The fluid then turns or transitions to a proximal direction to flow through the outer fluid flow channel 208 toward the handle H and motor M as described above. This embodiment thus does not require a physical deflection to turn the fluid flow at the desired location within the cooling mechanism 200. Instead, the fluid flow direction responds to and may be modified by a pressure differential between the first and second fluid pumps 218, 220.

Thus, in this embodiment the transition of the fluid flow direction from distal (through the outer fluid flow channel 208) to proximal (through the inner fluid flow channel 210) is induced solely by a pressure differential between the first fluid pump 218 and the second fluid pump 220. Here, the pressure P2 generated by the second fluid pump 220 may be greater than the pressure P1 generated by the first fluid pump 218 in order to induce the desired fluid flow pathway.

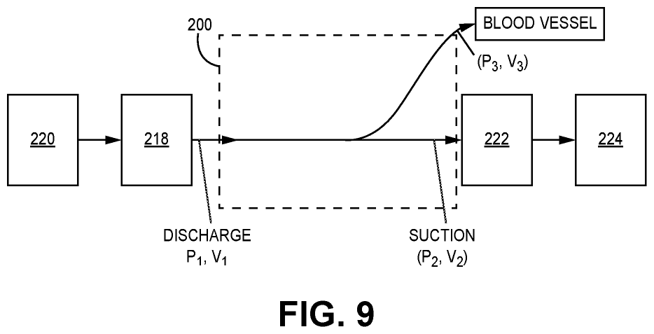

FIG. 9 illustrates a block diagram wherein the fluid deflection surface 207 is absent within the cooling mechanism 200. Here, the first fluid pump 218 generates a discharge or inflow pressure P1 and inflow volume V1 and the second fluid pump 222 generates a suction or outflow pressure P2 and outflow volume V2. A portion or fraction of the fluid within the cooling mechanism 200 is removed from the cooling mechanism 200 through at least one aperture Ap defined through the fluid deflection surface 207 to prevent blood inflow into the cooling mechanism 200 and handle H and/or motor M. The fluid that passes through the at least one aperture Ap flows through the impeller and out of outlet apertures as shown in FIG. 5A and into a blood vessel.

In some embodiments, the combination of the presence of the fluid deflection surface 207 and the discharge pressure of the first fluid pump 218 and the suction pressure of the second fluid pump 220 work together to ensure that the fluid flows in the desired pathway through the cooling mechanism 200 as described above. In these embodiments, a portion of the fluid may, as also discussed above, flow through at least one aperture Ap defined through the fluid deflection surface 207 to prevent blood inflow into the cooling mechanism 200 and handle H and/or motor.

Thus, the actively pumped fluid functions to cool the drive shaft as it rotates and heats to the frictional forces, and to cool the motor M as it operates.

A preferred cooling fluid is dextrose in water (DSW) and 5% DSW is more preferred. The artisan will recognize that other cooling fluids such as water or saline or other fluids may also be used with good cooling results.

With continued reference to FIG. 5A, and referring now to FIG. 5B, an alternate flow path may be used for cooling mechanism 200′. The primary difference between FIGS. 5A and 5B is that the inflow channel 202 is in fluid communication with the inner fluid flow channel 210. As a result that the fluid flow within the inner fluid flow channel 210 and the outer flow channel 208 are reversed in the embodiment of FIG. 5B as compared with the embodiment shown in FIG. 5A. Thus, in FIG. 5B, the fluid is shown as flowing distally through the inner fluid flow channel 210 until reaching the deflection surface 207 where the fluid is deflected into a proximal direction.

The fluid then flows proximally through the outer flow channel 208 and across the outer surface of the motor M. As described above, a portion of the flowing fluid may pass through one or more apertures Ap defined through the fluid deflection surface 207.

In other embodiments, a plurality of flow channels may be provided.

### WORKING EXAMPLE

Table 1 below illustrates exemplary fluid flow rates obtained using an exemplary blood pump system, specifically a ventricular assist device (“pVAD”), as described above and shown in, e.g., FIG. 5A, comprising a first fluid flow pump 218 and a second fluid flow pump 222 and a fluid deflection surface with aperture(s) Ap.

TABLE 1
pVAD @ 50

| | Avg | St Dev | Avg | St. Dev | Avg | St Dev |
|---|---|---|---|---|---|---|
| RPM | Waste Flow | Waste Flow | Purge Flow | Purge Flow | Ingress | Ingress |
| 60 | 4.27 | 0.21 | 4.26 | 0.29 | −0.004 | 0.19 |

pVAD @ 51

| | Avg | St Dev | Avg | St. Dev | Avg | St Dev | Max | Min |
|---|---|---|---|---|---|---|---|---|
| RPM | Waste Flow | Waste Flow | Purge Flow | Purge Flow | Ingress | Ingress | Ingress | Ingress |
| 20 | 1.73 | 0.05 | 1.85 | 0.07 | 0.120 | 0.09 | 0.27 | −0.01 |

pVAD @ 54

| | Avg | St Dev | Avg | St. Dev | Avg | St Dev | Max | Min |
|---|---|---|---|---|---|---|---|---|
| RPM | Waste Flow | Waste Flow | Purge Flow | Purge Flow | Ingress | Ingress | Ingress | Ingress |
| 20 | 1.57 | 0.06 | 1.77 | 0.06 | 0.200 | 0.08 | 0.35 | 0.09 |

The data corresponding with pVAD @ S0 indicates that the drive shaft is not rotating and the system air is being purged by pumping the first fluid pump (purge flow in Table 1) at 60 RPM. The waste flow and the purge flow are substantially the same. In other words, there is very little to no fluid flow that moves through the one or more apertures (Ap) to the patient's blood vessel as described above.

Next, with the pVAD rotating at rotational speed S1, and the first and second pumps 218, 222 operating at 20 RPM, the purge flow at the first fluid flow pump 218 is higher than the waste flow flowing out of the system at the second fluid flow pump 222. The difference between the two flow rates is the fluid flow that moves out of the system through the at least one aperture (A) and into the patient's blood vessel as described above.

With the pVAD rotating at rotational speed S4 (higher speed than S1), and the first and second pumps 218, 222 operating at 20 RPM, the purge flow at the first fluid flow pump 218 is higher than the waste flow flowing out of the system at the second fluid flow pump 222. The difference between the two flow rates is the fluid flow that moves out of the system through the at least one aperture (A) and into the patient's blood vessel as described above.

Thus, the flow rate of fluid that leaves the system, and enters the patient's blood vessel, through the one or more apertures Ap may be described as the difference between the flow rate generated by the first fluid pump 218 and the flow rate generated by the second fluid pump 222.

The description of the invention and is as set forth herein is illustrative and is not intended to limit the scope of the invention. Features of various embodiments may be combined with other embodiments within the contemplation of this invention. Variations and modifications of the embodiments disclosed herein are possible and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

1. A blood pump system comprising:
a handle enclosing an electric motor;
a drive shaft in operative engagement with the electric motor and with a blood pump impeller connected at a distal portion of the drive shaft and so as to be rotatable by the drive shaft and electric motor;
an inner sheath surrounding part of the drive shaft and defining a drive shaft lumen therethrough and comprising a distal end, wherein the distal portion of the drive shaft is not surrounded by the inner sheath;
an outer sheath surrounding, and radially spaced from, the inner sheath, and extending distally beyond a distal end of the inner sheath, wherein the outer sheath is at least partially closed at a distal end to form a fluid deflection surface, and wherein the distal end of the inner sheath terminates at a location that is proximal to the fluid deflection surface;
wherein an outer fluid flow channel is defined by a radial spacing between the outer sheath and the inner sheath, and an inner fluid flow channel is defined by a radial spacing between the inner sheath and the drive shaft;
and further comprising a first fluid pump in fluid communication with a fluid reservoir and with the outer fluid flow channel, wherein the first fluid pump is configured to actively pump fluid from the fluid reservoir through the outer fluid flow channel in a distal direction, and the fluid deflection surface is configured to deflect at least some of the fluid into the inner fluid flow channel such that the fluid flows in a proximal direction along the drive shaft, and out of the system.

2. The blood pump system of claim 2, further comprising a second fluid pump operatively connected with the inner fluid flow channel to actively pump the fluid out of the system.

3. The blood pump of claim 1, further comprising an inflow channel in fluid communication with the outer fluid flow channel and configured to receive actively pumped fluid pumped from the reservoir by the first fluid pump.

4. The blood pump of claim 1, wherein the fluid deflection surface is located proximal to the impeller.

5. The blood pump of claim 1, wherein the fluid deflection surface further defines one or more apertures therethrough.

6. The blood pump of claim 5, wherein a portion of the fluid pumped through the outer flow channel is in fluid communication with the impeller by way of the the one or more apertures.

7. (canceled)

8. The blood pump of claim 6, wherein the impeller is configured to rotate and move the fluid that is pumped through the one or more apertures into a patient's blood vessel.

9. The blood pump of claim 1, further comprising a housing surrounding the electric motor and defining a plurality of fluid guide apertures that are in fluid communication with, and configured to receive the fluid flowing from, the inner fluid channel and to direct the fluid flowing from the inner fluid channel across the outer surface of the electric motor.

10. (canceled)

11. The blood pump of claim 9, wherein the fluid guide apertures are configured to flow the received fluid across the outer surface of the electric motor in a helical flow pattern.

12. (canceled)

13. (canceled)

14. The blood pump of claim 1, further comprising at least one sensor that is operationally connected with the first fluid pump, the at least one sensor selected from a group consisting of one or more of: a pressure sensor, a temperature sensor, an optical clarity sensor, an osmolarity sensor, a fluid resistance sensor and a conductivity sensor.

15. The blood pump of claim 1, further comprising at least one sensor that is operationally connected with the second fluid pump, the at least one sensor selected from a group consisting of one or more of: a pressure sensor, a temperature sensor, an optical clarity sensor, an osmolarity sensor, a fluid resistance sensor and a conductivity sensor.

16. The blood pump of claim 9, further comprising at least one sensor that is operationally located within a flow path of fluid that is defined by at least one of the plurality of fluid guide apertures, the at least one sensor selected from a group consisting of one or more of: a pressure sensor, a temperature sensor, an optical clarity sensor, an osmolarity sensor, a fluid resistance sensor, and a conductivity sensor.

17.-38. (canceled)

39. A method for cooling a blood pump device comprising a rotational drive shaft and electric motor, wherein a proximal end of the rotational drive shaft is rotationally connected to the electric motor and a distal end of the rotational drive shaft is connected with a rotatable impeller, comprising:
providing a fluid reservoir and a first pump in fluid communication with the fluid reservoir;
providing an inner sheath surrounding part of the drive shaft and defining a drive shaft lumen therethrough, wherein a distal portion of the drive shaft is not surrounded by the inner sheath;
providing an outer sheath surrounding, and radially spaced from, the inner sheath, and extending distally beyond a distal end of the inner sheath, wherein the outer sheath is closed at a distal end;
providing an outer fluid flow channel defined by a radial spacing between the outer sheath and the inner sheath;
providing an inner fluid flow channel defined by a radial spacing between the inner sheath and the drive shaft, wherein a distal end of the outer fluid channel and a distal end of the inner fluid channel are in fluid communication;
providing a first fluid pump in fluid communication with a fluid reservoir and with the outer fluid flow channel; and
providing a second fluid pump in fluid communication with the inner fluid flow channel; and
actively pumping the fluid, by the first fluid pump and by the second fluid pump, from the fluid reservoir, through the outer fluid flow channel in a distal direction, through the inner fluid flow channel in a proximal direction, across or around an outer surface of the electric motor, and out of the system,
wherein a portion of the fluid within the outer fluid flow channel is pumped toward the impeller.

40. The method of claim 39, wherein the fluid flowing through the inner fluid channel is configured to remove heat generated by a rotating drive shaft.

41. The method of claim 39, wherein the fluid flowing across or around an outer surface of the electric motor is configured to remove heat generated by the electric motor.

42. A method for cooling a blood pump device comprising a rotational drive shaft and electric motor, wherein a proximal end of the rotational drive shaft is rotationally connected to the electric motor and a distal end of the rotational drive shaft is connected with a rotatable impeller, wherein an inner sheath surrounds part of the drive shaft and defines a drive shaft lumen, an outer sheath surrounds and is radially spaced from the inner sheath and extends distally beyond a distal end of the inner sheath, and the outer sheath is at least partially closed at a distal end thereof, the radial spacing between the outer sheath and the inner sheath defining a first fluid flow channel, and the radial spacing between the inner sheath and the drive shaft defining a second fluid flow channel, the method comprising the steps of:
creating a first fluid flow from a fluid reservoir to and distally through the first fluid flow channel to the distal end of outer sheath;
deflecting at least a portion of the first fluid flow toward the second fluid flow channel; and
creating a second fluid flow from the distal end of the outer sheath flowing proximally through the second fluid flow channel.

43. The method of claim 42, wherein the first fluid flow is created by a first fluid pump and the second fluid flow is created by a second fluid pump.

44. The method of claim 42, wherein a portion of the first fluid flow is discharged from first fluid flow channel by way of one or more apertures through the at least partially closed distal end of the outer sheath so as to be in fluid communication with the impeller by way of the one or more apertures.

45. The method of claim 44, wherein the impeller is configured to rotate and move the fluid that is discharged through the one or more apertures into a patient's blood vessel.
